(19) United States
(12) Patent Application Publication
(10) Pub. No.: US 2015/0173717 A1
(43) Pub. Date: Jun. 25, 2015

(54) ULTRASONIC DIAGNOSTIC DEVICE

(71) Applicant: GE MEDICAL SYSTEMS GLOBAL TECHNOLOGY COMPANY, LLC, Waukesha, WI (US)

(72) Inventor: Shunichiro Tanigawa, Tokyo (JP)

(73) Assignee: GE MEDICAL SYSTEMS GLOBAL TECHNOLOGY COMPANY, LLC, Waukesha, WI (US)

(21) Appl. No.: 14/576,567

(22) Filed: Dec. 19, 2014

(30) Foreign Application Priority Data
Dec. 24, 2013 (JP) 2013265015

(51) Int. Cl. A61B 8/00; A61B 8/08; A61B 8/14

(52) U.S. Cl. CPC A61B 8/463; A61B 8/14; A61B 8/54; A61B 8/08; A61B 8/5223; A61B 8/5207

(57) ABSTRACT

An ultrasonic diagnostic device is provided. The device includes a transmission control unit configured to generate a first ultrasonic wave and a second ultrasonic wave for detecting movement of a physical object in a biological tissue, wherein values of transmission parameters of repetitively transmitted first ultrasonic waves that are relevant to movement of the physical object are different from one another. The device further includes a decision unit configured to determine presence or absence of movement of the physical object, and a display image control unit configured to generate an image according to the value of the transmission parameter of the first ultrasonic wave in a case where the decision unit decides that movement is present, or a predetermined value in a case where the decision unit decides that movement is absent and the value of the transmission parameter of first ultrasonic wave has reached the predetermined value.

### CROSS REFERENCE TO RELATED APPLICATIONS

This application claims the benefit of Japanese Patent Application No. 2013-265015 filed Dec. 24, 2013, which is hereby incorporated by reference in its entirety.

### BACKGROUND

The present invention relates to an ultrasonic diagnostic device that displays an image that makes tissue characterizations distinguishable.

In an ultrasonic diagnostic device, an ultrasonic image such as a B-mode image or the like is created on the basis of an echo signal obtained by performing transmission of an ultrasonic wave to a test object. Then, a diagnosis for presence or absence of, for example, a mass is made on the basis of this ultrasonic image (see, for example, Japanese Patent Application Laid-Open No. 2004-41617).

Incidentally, there are cases when it is difficult to distinguish whether it is a mass or a tissue characterization other than that through simple observation of the B-mode image. For example, in the mammary gland, a concentrated cyst is the one that moisture in a secretion of the mammary gland is absorbed and fat content and so forth are left in large quantities. There are cases when it is difficult to distinguish such a concentrated cyst from the mass on the B-mode image. On the other hand, there are cases when even the mass looks like the cyst on the B-mode image. As described above, there are cases when distinction of the mass from the concentrated cyst is difficult on the B-mode image. Besides this, in the liver, distinction of a hepatic hemangioma from a hepatocarcinoma on the B-mode image is difficult.

### BRIEF DESCRIPTION

A physical object moves or does not move in accordance with the tissue characterization in the biological tissue to which the ultrasonic wave has been transmitted. Specifically, in a first aspect, an ultrasonic diagnostic device is provided. The ultrasonic diagnostic device is characterized by including a transmission control unit that makes a first ultrasonic wave and a second ultrasonic wave for detecting movement of a physical object in a biological tissue induced by the first ultrasonic wave transmit from an ultrasonic probe to the biological tissue alternately and repetitively, wherein values of transmission parameters of the aforementioned repetitively transmitted first ultrasonic wave, that is, the values of the transmission parameters relevant to movement of the aforementioned physical object are mutually different, a decision unit that decides presence or absence of movement of the aforementioned physical object on the basis of an echo signal obtained by transmission of the second ultrasonic wave for every transmission of the aforementioned second ultrasonic wave corresponding to the aforementioned first ultrasonic wave and a display image control unit that makes an image according to the value of the transmission parameter of the aforementioned first ultrasonic wave in a case where it has been decided that movement of the aforementioned physical object is present by the decision unit or the predetermined value in a case where it has been decided that movement of the aforementioned physical object is absent by the aforementioned decision unit and the value of the transmission parameter of the aforementioned first ultrasonic wave has reached the predetermined value for terminating transmission of the aforementioned first ultrasonic wave display.

Here, that one transmission of the aforementioned first ultrasonic wave and a plurality of transmissions of the aforementioned second ultrasonic wave are repeated a plurality of times is included in that the aforementioned transmission control unit makes the aforementioned first ultrasonic wave and the aforementioned second ultrasonic wave transmit alternately and repetitively, in addition to that one transmission of the aforementioned first ultrasonic wave and one transmission of the aforementioned second ultrasonic wave are repeated a plurality of times.

In a second aspect, an ultrasonic diagnostic device is provided. The ultrasonic diagnostic device is characterized by including a transmission control unit that makes a first ultrasonic wave transmit from an ultrasonic probe to a biological tissue and makes a second ultrasonic wave transmit from the aforementioned ultrasonic probe after this first ultrasonic wave has been transmitted, a detection unit that detects presence or absence of movement of a physical object in the aforementioned biological tissue by the aforementioned first ultrasonic in a two-dimensional region on the basis of an echo signal obtained by transmission of the aforementioned second ultrasonic wave, an evaluation unit that performs an evaluation for a tissue characterization regarding a part of interest of the aforementioned biological tissue on the basis of detection by this detection unit and a display image control unit that makes an image according to the evaluation by this evaluation unit display.

According to the first aspect, the image according to the value of the transmission parameter of the aforementioned first ultrasonic wave in the case where it has been decided that movement of the aforementioned physical object is present by the decision unit or the predetermined value in the case where it has been decided that movement of the aforementioned physical object is absent by the aforementioned decision unit and the value of the transmission parameter of the aforementioned first ultrasonic wave has reached the predetermined value for terminating transmission of this first ultrasonic wave is displayed. Therefore, in a case where movement of the physical object is detected or not detected due to a difference in tissue characterization in the aforementioned biological tissue to which the aforementioned first ultrasonic wave has been transmitted, the tissue characterization can be distinguished from another by the aforementioned image.

According to the second aspect, the evaluation for the tissue characterization regarding the part of interest of the aforementioned biological tissue is performed on the basis of detection of movement of the physical object in the two-dimensional region in the aforementioned biological tissue by the aforementioned first ultrasonic wave and the image according to this evaluation is displayed. Therefore, in the case where movement of the physical object is detected or not detected due to the difference in tissue characterization, the tissue characterization can be distinguished from another by the aforementioned image.

### BRIEF DESCRIPTION OF THE DRAWINGS

FIG. 1 is a block diagram showing a schematic configuration of an exemplary ultrasonic diagnostic device.

FIG. 2 is a block diagram showing a configuration of an echo data processing unit.

FIG. 3 is a block diagram showing a configuration of a display control unit.

FIG. 4 is a flowchart showing an operation of a first embodiment.

FIG. 5 is a diagram showing a display unit that a detection region has been set on a B-mode image.

FIG. 6 is an explanatory diagram showing flow of a structure in a cyst.

FIG. 7 is a diagram showing a display unit that a color image has been displayed.

FIG. 8 is a block diagram showing a configuration of a display control unit in a second embodiment.

FIG. 9 is a flowchart showing an operation of the second embodiment.

FIG. 10 is a block diagram showing a configuration of a display control unit in a third embodiment.

FIG. 11 is a flowchart showing an operation of the third embodiment.

FIG. 12 is a diagram showing a configuration of another example of the echo data processing unit.

### DETAILED DESCRIPTION

In the following, exemplary embodiments will be described.

### First Embodiment

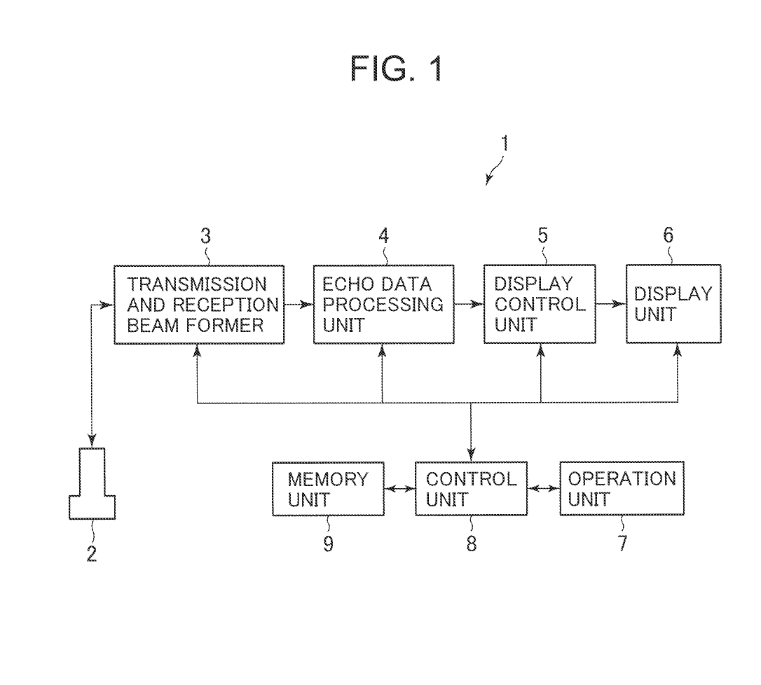

First, a first embodiment will be described. An ultrasonic diagnostic device 1 shown in FIG. 1 is provided with an ultrasonic probe 2, a transmission and reception beam former 3, an echo data processing unit 4, a display control unit 5, a display unit 6, an operation unit 7, a control unit 8 and a memory unit 9.

The aforementioned ultrasonic probe 2 transmits an ultrasonic wave to a biological tissue of a test object and receives an echo signal thereof. In the ultrasonic wave to be transmitted by this ultrasonic probe 2, a first ultrasonic wave and a second ultrasonic wave that detects movement of the biological tissue induced by this first ultrasonic wave are included. Details will be described later.

The aforementioned transmission and reception beam former 3 makes the aforementioned ultrasonic probe 2 drive on the basis of a control signal from the aforementioned control unit 8 and makes it transmit the aforementioned first ultrasonic and the aforementioned second ultrasonic wave having predetermined transmission parameters. In addition, the transmission and reception beam former 3 performs signal processing such as phasing addition processing and so forth on the echo signal of the ultrasonic wave. The aforementioned transmission and reception beam former 3 and the aforementioned control unit 8 are one example of an embodiment of a transmission control unit.

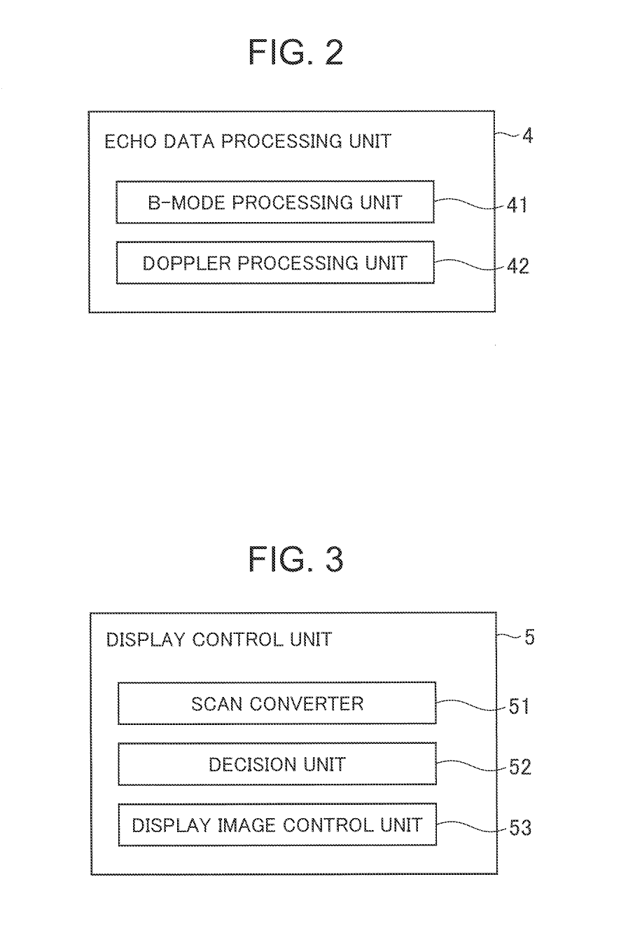

The aforementioned echo data processing unit 4 has a B-mode processing unit 41 and a Doppler processing unit 42 as shown in FIG. 2. The aforementioned B-mode processing unit 41 performs B-mode processing such as logarithmic compression processing, envelope detection processing and so forth on echo data output from the aforementioned transmission and reception beam former 3 to create B-mode data.

In addition, the aforementioned Doppler processing unit 42 performs Doppler processing on the echo data output from the aforementioned transmission and reception beam former 3 to create Doppler data. The Doppler processing includes orthogonal detection processing, filtering and so forth.

The aforementioned Doppler processing unit 42 performs, for example, color Doppler processing for creating a color Doppler image. However, the aforementioned Doppler processing unit 42 may perform pulse Doppler processing for creating an image by a pulse Doppler method and may perform continuous wave Doppler processing for creating an image by a continuous wave Doppler method.

The aforementioned B-mode processing unit 41 and the aforementioned Doppler processing unit 42 perform the aforementioned B-mode processing and the aforementioned Doppler processing on the basis of the echo data obtained by transmission of the aforementioned second ultrasonic wave.

The aforementioned display control unit 5 has a scan converter 51, a decision unit 52 and a display image control unit 53 as shown in FIG. 3. The aforementioned scan converter 51 scan-converts the aforementioned B-mode data to create B-mode image data.

The aforementioned decision unit 52 decides presence or absence of movement of the physical object in the biological tissue by transmission of the first ultrasonic wave on the basis of the aforementioned Doppler data. Details will be described later. The aforementioned decision unit 52 is one example of an embodiment of a decision unit.

The aforementioned display unit 6 is an LCD (Liquid Crystal Display), an organic EL (Electro-Luminescence) display and so forth. Though not shown in the drawing in particular, the aforementioned operation unit 7 is configured by including a keyboard, a pointing device such as a trackball and so forth and others in order that an operator inputs instructions and information.

Though not shown in the drawing in particular, the aforementioned control unit 8 is configured by having a CPU (Central Processing Unit). This control unit 8 reads out a control program stored in the aforementioned memory unit 9 and makes it execute a function of each unit of the aforementioned ultrasonic diagnostic device 1. The aforementioned ultrasonic diagnostic device 1 is provided with a configuration as a computer.

The aforementioned memory unit 9 is an HDD (Hard Disk Drive) or a semiconductor memory such as a RAM (Random Access Memory), a ROM (Read Only Memory) and so forth.

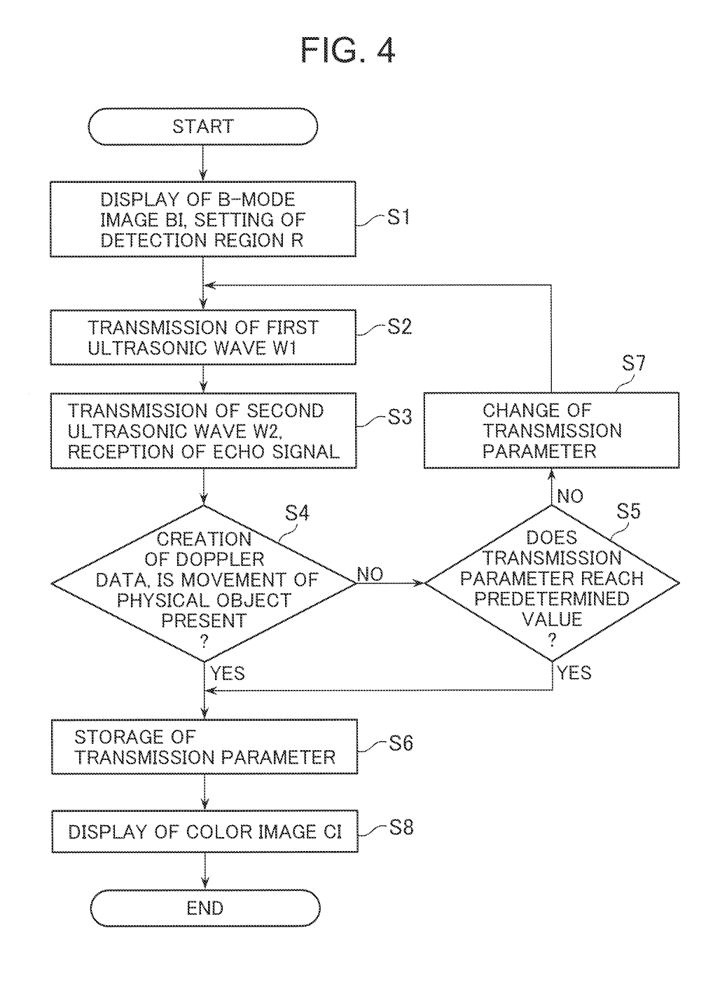

Next, the operation of the ultrasonic diagnostic device 1 of the present example will be described on the basis of a flowchart in FIG. 4. The flowchart in FIG. 4 is a flowchart for making an image for discriminating the tissue characterization of an observation object in the biological tissue of the test object display from another. In the present example, discrimination as to the tissue characterization is discrimination as to whether it is the mass or the concentrated cyst in a mamma and discrimination as to the degree of concentration of the cyst. In addition, discrimination as to the tissue characterization may be discrimination as to whether it is the mass or the hepatic hemangioma in the liver.

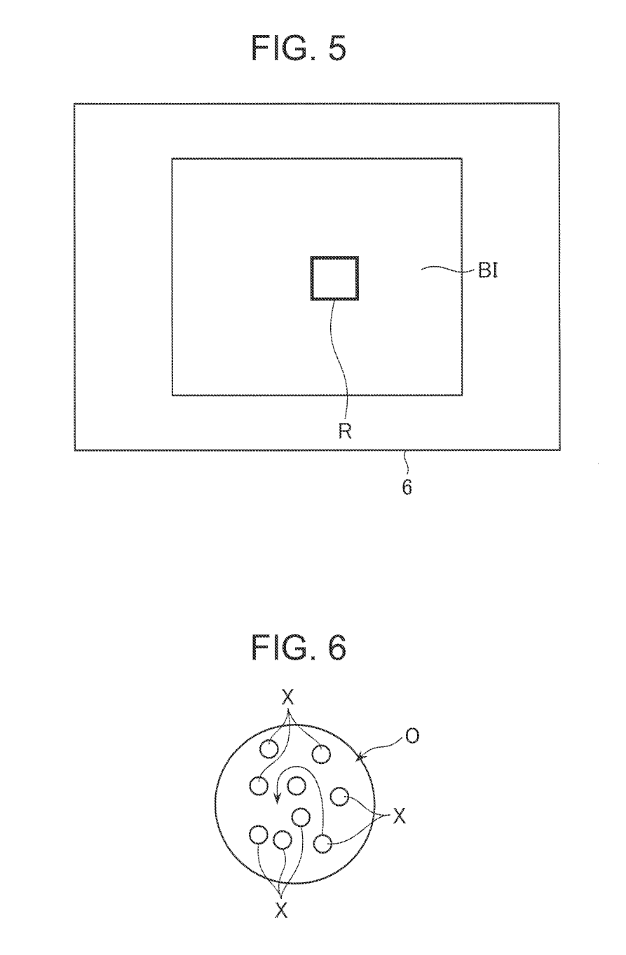

First, in step S1, the operator brings the aforementioned ultrasonic probe 2 into abutment on the body surface of the test object and performs transmission and reception of the ultrasonic wave to and from the biological tissue by this aforementioned ultrasonic probe 2 in order to make a B-mode image display. Thereby, a B-mode image BI is displayed on the aforementioned display unit 6 as shown in FIG. 5. In addition, the operator sets a detection region R on the aforementioned B-mode image BI by using the aforementioned operation unit 7.

The aforementioned detection region R is a region to be an object for detecting presence or absence of movement of the physical object in the biological tissue by transmission of the first ultrasonic wave as described later. The aforementioned detection region R is set on an object (an observation object) that a person who will make a diagnosis wishes to discriminate the tissue characterization thereof from another.

Next, in step S2, the operator performs input of a detection mode for movement of the physical object by the aforementioned operation unit 7. Thereby, the aforementioned control unit 8 makes a first ultrasonic wave W1 transmit from the aforementioned ultrasonic probe 2 to the biological tissue. This first ultrasonic wave W1 is a pulse wave having a value of a predetermined transmission parameter. The aforementioned first ultrasonic wave W1 is transmitted such that a beam thereof passes through the aforementioned observation object or the vicinity of the observation object.

Next, in step S3, the aforementioned control unit 8 makes a second ultrasonic wave W2 transmit from the aforementioned ultrasonic probe 2 to the biological tissue to which the aforementioned first ultrasonic wave W1 has been transmitted in the aforementioned step S2. Then, the aforementioned ultrasonic probe 2 receives the echo signal of the aforementioned second ultrasonic wave W2. This second ultrasonic wave W2 is an ultrasonic wave of a color Doppler mode. Transmission and reception of the aforementioned second ultrasonic wave W2 are performed so as to include the aforementioned detection region R. In this step S3, transmission of the aforementioned second ultrasonic wave W2 and reception of the echo signal thereof are performed by one frame. That is, in this step S3, transmission and reception of the aforementioned second ultrasonic wave are performed a plurality of times.

Next, in step S4, the aforementioned Doppler processing unit 42 creates the Doppler data on the basis of the echo signal obtained in the aforementioned step S3. Then, the aforementioned decision unit 52 decides presence or absence of movement of the physical object in the aforementioned detection region R on the basis of this Doppler data.

Decision by the aforementioned decision unit 52 and the physical object in the biological tissue will be described by giving a case where the aforementioned detection region R has been set on the observation object of the mamma by way of example. In FIG. 6, in a case where an observation object O was the cyst, a grained structure X is present in the cyst. This structure X is fat content and so forth and has fluidity. The aforementioned structure X flows by the aforementioned first ultrasonic wave W1, for example, as shown by an arrow (only movement of one structure X is shown). The aforementioned structure X is one example of the aforementioned physical object.

However, the fluidity of the structure X is varied depending on the degree of concentration of the cyst. Specifically, since the higher the degree of concentration of the cyst becomes (moisture is reduced), the higher the density of the structures X becomes, the fluidity of the structure X becomes lower. On the other hand, since the lower the degree of concentration of the cyst becomes (the moisture is increased), the lower the density of the structures X becomes, the fluidity of the structure X becomes higher.

Whether the structure X flows or not by transmission of the aforementioned first ultrasonic wave W1 is determined by two conditions of a state in the cyst relevant to the fluidity of the structure and values of the transmission parameters of the aforementioned first ultrasonic wave W1. The transmission parameters so called here are parameters relevant to flow of the structure by the ultrasonic wave and are such as a voltage (a transmission voltage) to be applied to an ultrasonic transducer upon transmission, a transmission frequency, a pulse length of the ultrasonic wave and so forth. Specifically, the higher the transmission voltage becomes, the lower the transmission frequency becomes or the longer the pulse length becomes, the more the ultrasonic wave can make the structure flow even when the fluidity of the structure is low. Conversely, the lower the transmission voltage becomes, the higher the transmission frequency becomes or the shorter the pulse length becomes, the more the ultrasonic wave cannot make the structure flow unless the fluidity of the structure is high.

The aforementioned structure X is present in the aforementioned observation object O, and if this flows by the aforementioned first ultrasonic wave W1, flow velocity data and so forth indicating that the aforementioned structure X is flowing can be obtained as the aforementioned Doppler data. On the other hand, in a case where although the aforementioned structure X is present in the aforementioned observation object O, it is not flowing or, to begin with, the aforementioned structure X was not present in the aforementioned observation object O, the flow velocity data and so forth indicating that the aforementioned structure X is flowing cannot be obtained as the Doppler data. For example, the structure X that flows by the first ultrasonic wave W1 is not present in the mass and the flow velocity data cannot be obtained. Therefore, the aforementioned decision unit 52 decides presence or absence of movement of the physical object on the basis of the aforementioned Doppler data.

Incidentally, although a case where the aforementioned observation object is the cyst is shown in FIG. 6, the flow velocity data and so forth indicating that the hepatic hemangioma is moving can be obtained as the Doppler data also in a case where the hepatic hemangioma has moved by the first ultrasonic wave.

In the aforementioned step S4, in a case where it has been decided that movement of the physical object is present by the aforementioned decision unit 52 (“NO” in the aforementioned step S4), it proceeds to the process of step S5. On the other hand, in a case where it has been decided that movement of the physical object is absent by the aforementioned decision unit 52 (“YES” in the aforementioned step S4), it proceeds to the process of step S6.

In the aforementioned step S5, the aforementioned control unit 8 decides whether values of the transmission parameters relevant to movement of the physical object such as the transmission voltage, the transmission frequency, the pulse length and so forth in the transmission parameters of the aforementioned first ultrasonic wave W1 have reached predetermined values that have been set in advance. In this step S5, in a case where it has been decided that the values of the aforementioned transmission parameters do not reach the predetermined values so set in advance (“NO” in the aforementioned step S5), it proceeds to the process of step S7. On the other hand, in a case where it has been decided that the values of the aforementioned transmission parameters have reached the predetermined values so set in advance (“YES” in the aforementioned step S5), it proceeds to the process of step S6.

In the aforementioned step S7, the aforementioned control unit 8 changes the values of the transmission parameters relevant to movement of the physical object such as the transmission voltage, the transmission frequency, the pulse length and so forth in the transmission parameters of the first ultrasonic wave W1 to be transmitted the next. Specifically, the aforementioned control unit 8 changes the values of the transmission parameters so as to make movement of the physical object easier. For example, the aforementioned control unit 8 makes the transmission voltage higher than that upon transmission of the latest first ultrasonic wave W1. In addition, the aforementioned control unit 8 may make the transmission frequency lower than that upon transmission of the latest first ultrasonic wave W1. In addition, the aforementioned control unit 8 may set the values of the transmission parameters such that the pulse length becomes longer than that upon transmission of the latest first ultrasonic wave W1.

The values of the transmission parameters to be changed in the aforementioned step S7 may be stored in advance. In this case, the values of the transmission parameters are stored together with the order in which they are set. For example, V1, V2, . . . , V(N−1) and VN (N: a natural number) are stored to be changed in this order as the transmission voltage (V1<V2, . . . , V(N−1)<VN). In addition, F1, F2, . . . ,F(N−1) and FN (N: a natural number) are stored to be changed in this order as the transmission frequency (F1>F2, . . . , F(N−1)>FN). In addition, the values of the transmission parameter corresponding to the respective pulse lengths are stored such that the pulse length is changed in order of L1, L2, . . . , L(N−1) and LN (N: a natural number) (L1<L2, . . . , L(N−1)<LN)

It is desirable that the values of the transmission parameters (the aforementioned transmission voltage V1, the aforementioned transmission frequency F1 and the aforementioned pulse length L1) of the aforementioned first ultrasonic wave W1 to be firstly transmitted be set to values that in a case where the aforementioned observation object O is the cyst, the physical object moves in a case where the degree of concentration is low to some extent and the physical object does not move in a case where the degree of concentration is high to some extent. Since movement of the physical object is detected or not detected in accordance with the degree of concentration of the cyst by setting them to such values, an image according to the degree of concentration of the cyst can be obtained as a later described color image CI.

When the values of the transmission parameters are changed in the aforementioned step S7, it returns to the aforementioned step S2. In this step S2, transmission of the first ultrasonic wave W1 having the changed values of the transmission parameters is performed. Then, the processes of the aforementioned steps S2, S3, S4, S5 and S7 are repeated.

Incidentally, the predetermined values so set in advance that are the values of the transmission parameters serving as standards of decision are the tail-end values (the values of the transmission parameters for terminating transmission of the first ultrasonic wave W1) of the transmission parameters in the values of the transmission parameters to be changed in the aforementioned step S7. For example, the predetermined value so set in advance in the transmission voltage is the aforementioned transmission voltage VN, the predetermined value so set in advance in the transmission frequency is the aforementioned transmission frequency FN. In addition, the predetermined value so set in advance in the pulse length is the aforementioned pulse length LN.

In a case where it has been decided that movement of the physical object is present in the aforementioned step S4, the values of the transmission parameters (the transmission voltage, the transmission frequency, the pulse length and so forth) relevant to movement of the physical object in the values of the transmission parameters of the latest first ultrasonic wave are stored into the aforementioned memory unit 9 in step S6.

In addition, in a case where it has been decided that the values of the transmission parameters reach the predetermined values in the aforementioned step S5, looping of the processes of the aforementioned steps S2, S3, S4, S5 and S7 is terminated and it proceeds to the process of the aforementioned step S6. In this step S6, the predetermined values so set in advance (the aforementioned transmission voltage VN, the transmission frequency FN and the aforementioned pulse length LN) that are the values of the transmission parameters serving as the standards of decision in the aforementioned step S5 are stored into the aforementioned memory unit 9. The predetermined value so set in advance that is the value of the transmission parameter serving as the standard of decision in the aforementioned step S5 and to be stored in the aforementioned step S6 is one example of an embodiment of that predetermined value in the case where it has been decided that movement of the aforementioned physical object is absent by the decision unit and the value of the transmission parameter of the first ultrasonic wave has reached the predetermined value for terminating transmission of the first ultrasonic wave.

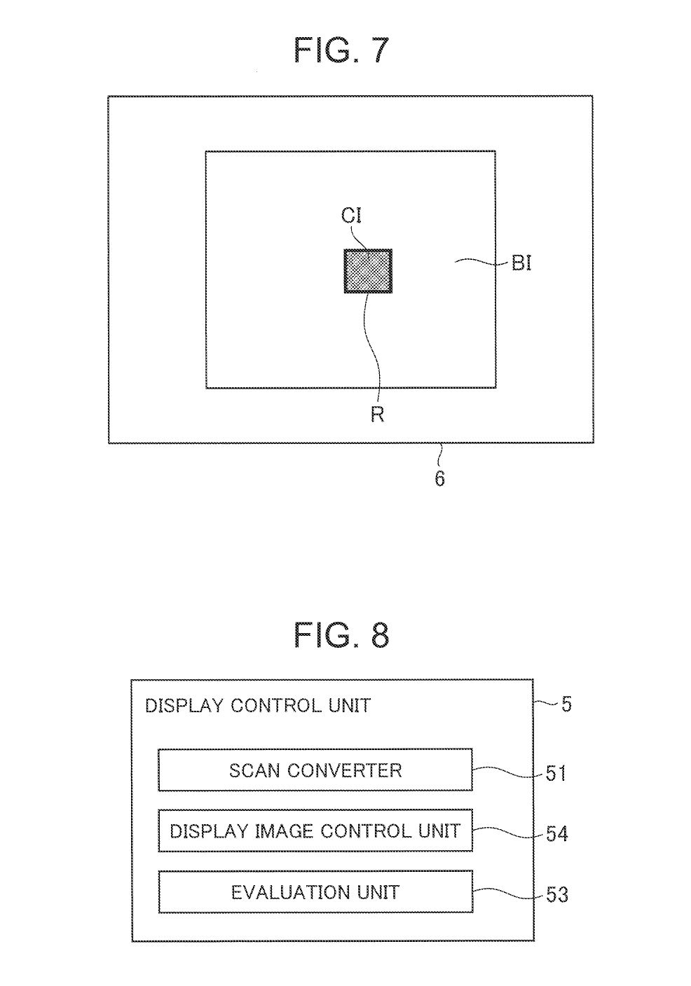

When storage of the values of the transmission parameters is performed in step S6, it proceeds to the process of step S8. In this step S8, the aforementioned display image control unit 53 makes the color image CI having a color according to the values of the transmission parameters stored in the aforementioned step S6 display on the aforementioned display unit 6 as shown in FIG. 7.

The aforementioned color image CI is displayed in the aforementioned detection region R. However, it is not limited to this. The aforementioned color image CI may be displayed so as to overlay the B-mode image BI (in a state that the background B-mode image BI is not seen through it). In addition, the aforementioned color image CI may be displayed by being synthesized with the B-mode image BI (in a state that the background B-mode image BI is seen through it).

In a case where, for example, V1 to VN are stored as the transmission voltage, Fl to FN are stored as the transmission frequency and L1 to LN are stored as the pulse length, the color of the aforementioned color image CI is set to N in accordance with the number N of the aforementioned parameters so stored.

According to the first embodiment, since the value of the transmission parameter is changed in turn so as to make movement of the physical object easier and the color image CI having the color according to the value of the transmission parameter of the first ultrasonic wave W1 when movement of the physical object has been detected or that predetermined value in the case where it has been decided that movement of the physical object is absent and the value of the transmission parameter of the first ultrasonic wave W1 has reached the predetermined value for terminating transmission of the first ultrasonic wave W1 is displayed, the tissue characterization can be distinguished from another in accordance with the color of this color image CI. Specifically, whether the observation object is the cyst or the mass can be seen from the color of the aforementioned color image CI and in addition in a case where the observation object was the cyst, the degree of concentration of the cyst can be seen. In addition, whether it is the hepatic hemangioma or the mass can be seen.

Incidentally, in the first embodiment, the image according to the value of the transmission parameter of the first ultrasonic wave W1 in the case where it has been decided that movement of the aforementioned physical object is present or the value of the transmission parameter for terminating transmission of the first ultrasonic wave W1 may be displayed on the aforementioned display unit 6. For example, the aforementioned display image control unit 53 may make a numeral of 1 to N according to the aforementioned transmission parameter display on the aforementioned display unit 6 in place of the aforementioned color image CI. In this case, the numerical image is one example of an embodiment of an image to be displayed by a display image control unit.

In the first embodiment, the processes of steps S1 to S7 may be repeated a plurality of times and the value of the transmission parameter may be stored a plurality of times in the aforementioned step S6. In this case, the color image CI based on the plurality of stored values of the transmission parameter may be displayed. For example, an average value of the plurality of the stored transmission parameters may be calculated and the color image CI according to this average value may be displayed.

### Second Embodiment

Next, an ultrasonic diagnostic device of a second embodiment will be described. In the following, matters different from those in the first embodiment will be described.

As shown in FIG. 8, in the second embodiment, the aforementioned display control unit 5 has an evaluation unit 54 in addition to the aforementioned scan converter 51 and the aforementioned display image control unit 53.

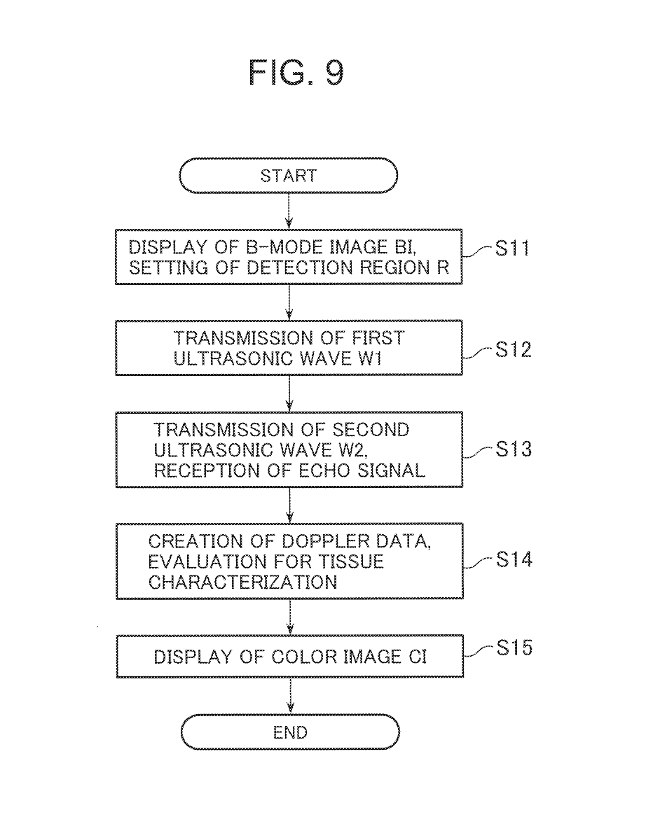

The operation of the second embodiment will be described on the basis of a flowchart in FIG. 9. Steps S11 to S13 are the same as the aforementioned steps S1 to S3 in the first embodiment. However, the value of the transmission parameter of the first ultrasonic wave W1 to be transmitted in the aforementioned step S12 is set to a value that makes it possible to move the physical object. For example, the value of the transmission parameter of this first ultrasonic wave W1 is set to the value that makes it possible to move the physical object in the cyst that is high in degree of concentration to some extent and is difficult in distinction from the mass on the B-mode image.

In step S14, similarly to the aforementioned step S4 of the first embodiment, the aforementioned Doppler processing unit 42 creates the Doppler data on the basis of the echo signal obtained in the aforementioned step S3. Here, the Doppler data is the color Doppler data. If the physical object is moving by the aforementioned first ultrasonic wave W1, the color Doppler data can be obtained. The aforementioned Doppler processing unit 42 is one example of an embodiment of a detection unit that detects presence or absence of movement of the physical object in the two-dimensional region. In addition, the aforementioned evaluation unit 54 performs an evaluation for the tissue characterization regarding the part of interest of the aforementioned biological tissue on the basis of the aforementioned Doppler data. The aforementioned evaluation unit 54 is one example of an embodiment of an evaluation unit.

The evaluation by the aforementioned evaluation unit 54 will be described. For example, the aforementioned evaluation unit 54 performs the evaluation according to the size of a region from which the aforementioned Doppler data has been obtained in the aforementioned detection region R. The size of the region from which the aforementioned Doppler data has been obtained is, for example, a number of pixels from which the Doppler data has been obtained, a ratio of the number of pixels from which the Doppler data has been obtained to the total number of pixels in the aforementioned measurement region R or the like. The aforementioned evaluation unit 54 determines a level relevant to the tissue characterization in accordance with the size of the region from which the aforementioned Doppler data has been obtained. This level is, for example, a level 1 to N (where N is a natural number larger than 1).

For example, the levels 1 to N indicate the degree of concentration of the cyst and distinction as to whether it is the cyst or the mass. In this case, the level 1 indicates that the degree of concentration of the cyst is the lowest (the degree of fluidity is large) and they indicate that the more the numeral of the level is increased to the level 2, 3, . . . , the higher the degree of concentration is. Then, the level N indicates that it is the mass. Accordingly, the more the number of pixels from which the Doppler data has been obtained is increased, the smaller the numerical value of the level becomes, on the other hand, the more the number of pixels from which the Doppler data has been obtained is reduced, the larger the numerical value of the level becomes. The level N is the level in a case where the Doppler data indicating the flow velocity and so forth is not obtained.

The aforementioned evaluation unit 54 may perform an evaluation according to a flow velocity value and a variance value of the flow velocity obtained on the basis of the aforementioned Doppler data. In this case, the aforementioned evaluation unit 54 calculates an average value of the flow velocity values and a variance value of the flow velocity of all pixels from which the Doppler data has been obtained in the aforementioned detection region R. Then, the aforementioned evaluation unit 54 determines the level 1 to N relevant to the tissue characterization in accordance with the aforementioned flow velocity value or the variance value. Specifically, since the larger the flow velocity value or the variance value is, the higher the fluidity of the physical body is, the numerical value of the level becomes smaller, on the other hand, since the smaller the flow velocity value or the variance value is, the lower the fluidity of the physical object is, the numerical value of the level becomes larger.

Next, in step S15, the aforementioned display image control unit 53 makes the color image CI having the color according to the level determined by the aforementioned evaluation unit 54 display on the aforementioned display unit 6.

According to the second embodiment, since the color image CI having the color according to the evaluation for the tissue characterization is displayed, the tissue characterization can be distinguished from another in accordance with the color of the aforementioned color image CI similarly to the first embodiment.

In the second embodiment, the aforementioned second ultrasonic wave W2 is an ultrasonic wave of a pulse Doppler mode or a continuous wave Doppler mode, and the aforementioned Doppler data may be Doppler data obtained by pulse Doppler processing or Doppler data obtained by continuous wave Doppler processing. In this case, the level 1 to N relevant to the tissue characterization is determined in accordance with the flow velocity value in the Doppler data.

In addition, in general, transmission and reception of the ultrasonic waves for obtaining the color Doppler data of one frame are performed a plurality of times for every sound ray and a plurality of flow velocities are obtained in each pixel. Therefore, the aforementioned evaluation unit 54 may calculate the average value of the variance values of the flow velocity of each pixel in the aforementioned detection region R to determine the aforementioned level 1 to N in accordance with the obtained average value.

### Third Embodiment

Next, an ultrasonic diagnostic device of a third embodiment will be described. In the following, the matters different from those in the first and second embodiments will be described.

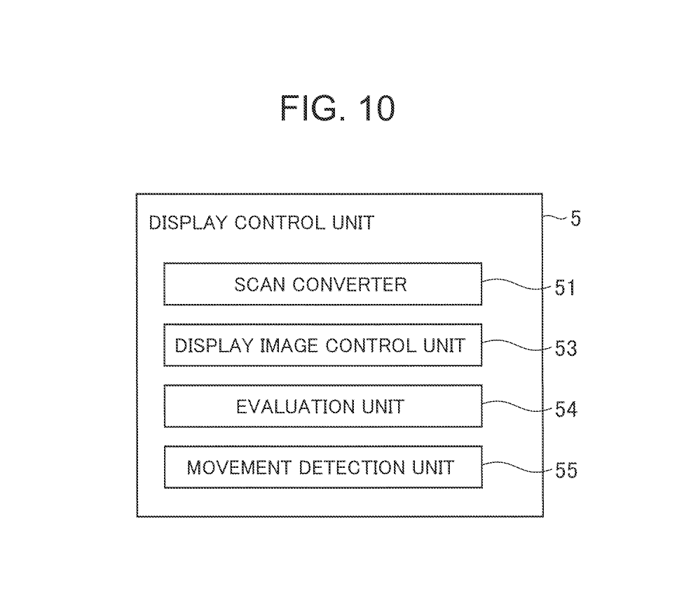

As shown in FIG. 10, in the third embodiment, the aforementioned display control unit 5 has a movement detection unit 55 in addition to the aforementioned scan converter 51, the aforementioned display image control unit 53 and the evaluation unit 54.

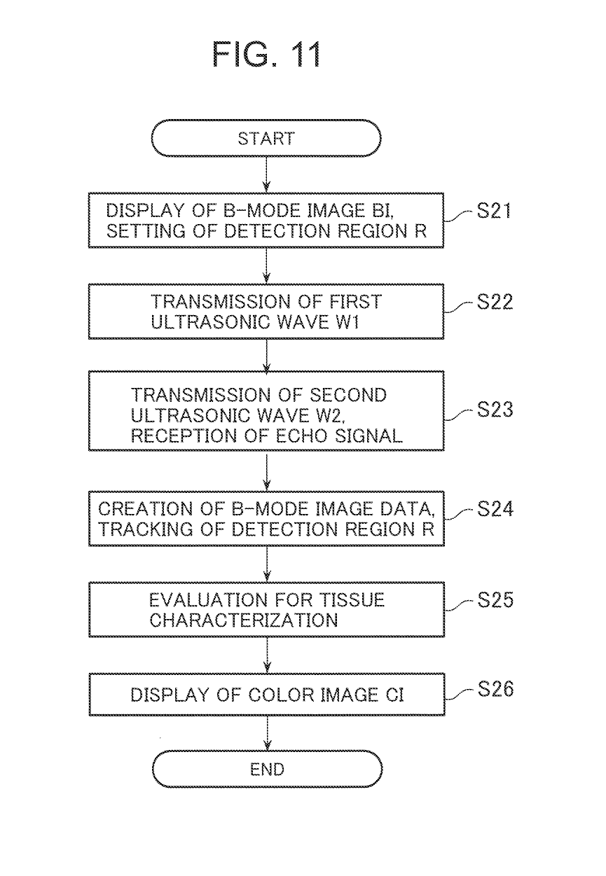

The operation of the third embodiment will be described on the basis of a flowchart in FIG. 11. Steps S21 and S22 are the same as the steps S1 and S2, and S11 and S12 in the aforementioned respective embodiments. The values of the transmission parameters of the first ultrasonic wave W1 in the aforementioned step S22 are the same as those in the step S12 of the aforementioned second embodiment.

In step S23, the ultrasonic wave for the B-mode is transmitted as the second ultrasonic wave W2 and the echo signal thereof is received. Next, in step S24, B-mode data and B-mode image data are created on the basis of the obtained echo signal. Then, the aforementioned movement detection unit 55 performs tracking on a speckle in the detection region R set in the aforementioned step S1. This tracking process is a process for tracking movement of the detection region R in association with movement of the physical object in the biological tissue by transmission of the aforementioned first ultrasonic wave W1. The aforementioned movement detection unit 55 is one example of the embodiment of the detection unit.

The aforementioned movement detection unit 55 performs a pattern matching process using correlation operation, for example, targeting on the B-mode image data of two frames and performs tracking on the speckle in the aforementioned detection region R.

Next, in step S25, the aforementioned evaluation unit 54 performs an evaluation for the tissue characterization regarding the part of interest of the biological tissue on the basis of a result of detection of movement of the aforementioned detection region R by the aforementioned movement detection unit 55. For example, the aforementioned evaluation unit 54 performs the evaluation according to a moving distance of the aforementioned detection region R. In this case, the aforementioned evaluation unit 54 determines the level 1 to N relevant to the tissue characterization in accordance with the magnitude of the aforementioned moving distance similarly to the aforementioned second embodiment. In the third embodiment, the larger the aforementioned moving distance is, the smaller the numerical value of the level becomes, on the other hand, the smaller the aforementioned moving distance is, the larger the numerical value of the level becomes. If the aforementioned moving distance is zero, the level N will be reached.

Next, in step S26, the aforementioned display image control unit 53 makes the color image CI having the color according to the level determined by the aforementioned evaluation unit 54 display on the aforementioned display unit 6 similarly to step S15 in the aforementioned second embodiment (see FIG. 7).

According to the third embodiment, since the color image CI having the color according to the evaluation for the tissue characterization is displayed similarly to the second embodiment, the tissue characterization can be distinguished from another in accordance with the color of the aforementioned color image CI.

Although the disclosure has been described by the aforementioned exemplary embodiments as mentioned above, it goes without saying that the systems and methods described herein can be modified in a variety of ways within a range not changing the gist thereof. For example, in the aforementioned second and third embodiments, the aforementioned display image control unit 53 may make a character of the aforementioned level 1 to N determined by the aforementioned evaluation unit 54 display on the display unit 6. In this case, the character image is one example of an embodiment of the image to be displayed by a display image control unit.

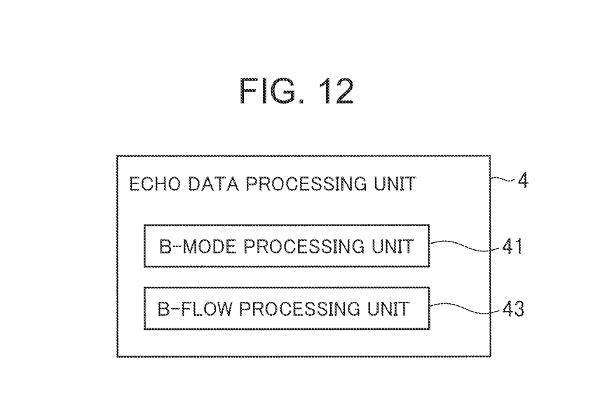

In addition, in the aforementioned each embodiment, the aforementioned second ultrasonic wave W2 may be an ultrasonic wave of not the Doppler mode but a B-flow mode. In this case, as shown in FIG. 12, the aforementioned echo data processing unit 4 has a B-flow processing unit 43 in place of the aforementioned Doppler processing unit 42. This B-flow processing unit 43 is one example of an embodiment of the detection unit. In the aforementioned first embodiment, the aforementioned decision unit 52 decides presence or absence of movement of the physical object on the basis of B-flow data obtained by the aforementioned B-flow processing unit 43. In addition, in the aforementioned second embodiment, it determines the level 1 to N relevant to the tissue characterization in accordance with the size of the region from which the aforementioned B-flow data has been obtained in the aforementioned detection region R. Further, in the aforementioned third embodiment, the aforementioned movement detection unit 55 performs speckle tracking of the aforementioned detection region R, targeting on the B-flow image data of two frames obtained by scan-converting the B-flow data by the scan converter 51. Then, the evaluation based on a result of detection of movement of the aforementioned detection region R is performed.

In addition, in the aforementioned second embodiment, transmission of the aforementioned first ultrasonic wave W1 and transmission of the ultrasonic wave W2 corresponding to it may be performed a plurality of times and the aforementioned evaluation unit 54 may perform the evaluation on the basis of the plurality of pieces of Doppler data or the plurality of pieces of B-flow data obtained by the plurality of these transmissions of the second ultrasonic wave W2. For example, the aforementioned evaluation unit 54 may calculate an average value of the sizes of the region from which the aforementioned Doppler data or the aforementioned B-flow data has been obtained, an average value of flow velocity values, an average value of variance values of the flow velocity, an average value of averages of the variance values of the flow velocity and may determine the aforementioned level 1 to N in accordance with this average.

In addition, in the aforementioned third embodiment, transmission of the aforementioned first ultrasonic wave W1 and transmission of the second ultrasonic wave W2 corresponding to it may be performed a plurality of times, the moving distance of the aforementioned detection region R may be calculated in plural by the aforementioned movement detection unit 55 and the aforementioned evaluation unit 54 may perform the evaluation on the basis of the plurality of moving distances. For example, the aforementioned evaluation unit 54 may calculate an average of the plurality of moving distances and may determine the aforementioned level 1 to N in accordance with this average.

1. An ultrasonic diagnostic device comprising:
a transmission control unit configured to generate a first ultrasonic wave and a second ultrasonic wave for detecting movement of a physical object in a biological tissue, the movement induced by transmitting the first ultrasonic wave from an ultrasonic probe to the biological tissue alternately and repetitively, wherein values of transmission parameters of the repetitively transmitted first ultrasonic wave that are relevant to movement of the physical object are different from one another;
a decision unit configured to determine presence or absence of movement of the physical object based on an echo signal obtained by transmission of the second ultrasonic wave for every transmission of the second ultrasonic wave corresponding to the first ultrasonic wave; and
a display image control unit configured to generate an image according to:
the value of the transmission parameter of the first ultrasonic wave in a case where the decision unit decides that movement of said physical object is present; or
a predetermined value in a case where the decision unit decides that movement of said physical object is absent and the value of the transmission parameter of first ultrasonic wave has reached the predetermined value that corresponds to terminating transmission of the first ultrasonic wave.

2. The ultrasonic diagnostic device defined in claim 1, wherein the second ultrasonic wave is an ultrasonic wave for a Doppler mode or a B-flow.

3. The ultrasonic diagnostic device defined in claim 1, further comprising:
a memory unit configured to store a plurality of at least any of:
the value of the transmission parameter of the first ultrasonic wave in the case where the decision unit decides that movement of said physical object is present; and
the predetermined value in the case where the decision unit decides that movement of said physical object is absent;
wherein the display image control unit is configured to generate the image based on the plurality of values stored in said memory unit.

4. The ultrasonic diagnostic device defined in claim 2, further comprising:
a memory unit configured to store a plurality of at least any of:
the value of the transmission parameter of the first ultrasonic wave in the case where the decision unit decides that movement of said physical object is present; and
the predetermined value in the case where the decision unit decides that movement of said physical object is absent;
wherein the display image control unit is configured to generate the image based on the plurality of values stored in said memory unit.

5. An ultrasonic diagnostic device comprising:
a transmission control unit configured to make a first ultrasonic wave transmit from an ultrasonic probe to a biological tissue and configured to make a second ultrasonic wave transmit from the ultrasonic probe after the first ultrasonic wave has been transmitted;
a detection unit configured to detect presence or absence of movement of a physical object in the biological tissue by the first ultrasonic in a two-dimensional region based on an echo signal obtained by transmission of the second ultrasonic wave;
an evaluation unit configured to perform an evaluation for a tissue characterization regarding a part of interest of the biological tissue, the evaluation performed based on the detection by the detection unit; and
a display image control unit configured to generate an image according to the evaluation by the evaluation unit display.

6. The ultrasonic diagnostic device defined in claim 5, wherein processing for detecting presence or absence of movement of the physical object by said detection unit is color Doppler processing or B-flow processing, and wherein said evaluation unit is configured to perform the evaluation according to a size of a first region from which Doppler data by the color Doppler processing has been obtained or second a region from which B-flow data by the B-flow processing has been obtained.

7. The ultrasonic diagnostic device defined in claim 5, wherein processing for detecting presence or absence of movement of the physical object by said detection unit is color Doppler processing, and wherein said evaluation unit is configured to perform the evaluation according to a speed of the physical object obtained by the color Doppler processing.

8. The ultrasonic diagnostic device defined in claim 5, wherein processing for detecting presence or absence of movement of the physical object by said detection unit is color Doppler processing, and wherein said evaluation unit is configured to perform the evaluation according to a variance of a speed of said physical object obtained by the color Doppler processing.

9. The ultrasonic diagnostic device defined in claim 5, wherein processing for detecting presence or absence of movement of the physical object by said detection unit is processing for tracking movement of the two-dimensional region in the biological tissue based on B-mode image data or B-flow image data created based on an echo signal by transmission of the second ultrasonic wave, and wherein said evaluation unit is configured to perform the evaluation based on a moving amount of the two-dimensional region.

10. The ultrasonic diagnostic device defined in claim 5, wherein said transmission control unit is configured to make the first ultrasonic wave and the second ultrasonic wave transmit a plurality of times, wherein said detection unit is configured to perform detection of presence or absence of movement of the physical object a plurality of times, and wherein said evaluation unit is configured to perform the evaluation based on the plurality of detections by said detection unit.

11. The ultrasonic diagnostic device defined in claim 6, wherein said transmission control unit is configured to make the first ultrasonic wave and the second ultrasonic wave transmit a plurality of times, wherein said detection unit is configured to perform detection of presence or absence of movement of the physical object a plurality of times, and wherein said evaluation unit is configured to perform the evaluation based on the plurality of detections by said detection unit.

12. The ultrasonic diagnostic device defined in claim 7, wherein said transmission control is configured to make the first ultrasonic wave and the second ultrasonic wave transmit a plurality of times, wherein said detection unit is configured to perform detection of presence or absence of movement of the physical object a plurality of times, and wherein said evaluation unit is configured to perform the evaluation based on the plurality of detections by said detection unit.

13. The ultrasonic diagnostic device defined in claim 8, wherein said transmission control unit is configured to make the first ultrasonic wave and the second ultrasonic wave transmit a plurality of times, wherein said detection unit is configured to perform detection of presence or absence of movement of the physical object a plurality of times, and wherein said evaluation unit is configured to perform the evaluation based on the plurality of detections by said detection unit.

14. The ultrasonic diagnostic device defined in claim 9, wherein said transmission control unit is configured to make the first ultrasonic wave and the second ultrasonic wave transmit a plurality of times, wherein said detection unit is configured to perform detection of presence or absence of movement of the physical object a plurality of times, and wherein said evaluation unit is configured to perform the evaluation based on the plurality of detections by said detection unit.

15. An ultrasonic diagnostic device comprising:
a transmission control unit configured to make a first ultrasonic wave transmit from an ultrasonic probe to a biological tissue and configured to make a second ultrasonic wave of a pulse Doppler mode or a continuous wave Doppler mode transmit from the ultrasonic probe after the first ultrasonic wave has been transmitted;
a Doppler data creation unit configured to create Doppler data based on an echo signal obtained by transmission of the second ultrasonic wave;
an evaluation unit configured to perform an evaluation for a tissue characterization regarding a part of interest of the biological tissue based on the Doppler data; and
a display image control unit configured to generate an image according to the evaluation by said evaluation unit.

16. The ultrasonic diagnostic device defined in claim 15, wherein said Doppler data creation unit is configured to create Doppler data using orthogonal detection processing.

17. The ultrasonic diagnostic device defined in claim 15, wherein said evaluation unit is configured to perform the evaluation based on a flow velocity value of the Doppler data.

18. The ultrasonic diagnostic device defined in claim 15, wherein said evaluation unit is configured to perform the evaluation based on a variance value of a flow velocity of the Doppler data.

19. The ultrasonic diagnostic device defined in claim 15, wherein said Doppler data creation unit is configured to create Doppler data using pulse Doppler processing.

20. The ultrasonic diagnostic device defined in claim 15, wherein said Doppler data creation unit is configured to create Doppler data using continuous wave Doppler processing.
